SEQUENCE LISTING (10 sequences)

SEQ ID NO: 1 - DNA, 20 bp, Artificial Sequence
  Description of Artificial Sequence Synthetic primer
gtgggcagcaaatgttgtaa

SEQ ID NO: 2 - DNA, 20 bp, Artificial Sequence
  Description of Artificial Sequence Synthetic primer
tcatcgacttccagagctga

SEQ ID NO: 3 - DNA, 20 bp, Artificial Sequence
  Description of Artificial Sequence Synthetic primer
accatgaagtgggtggaatc

SEQ ID NO: 4 - DNA, 20 bp, Artificial Sequence
  Description of Artificial Sequence Synthetic primer
tggtagccaggtcagctaaa

SEQ ID NO: 5 - DNA, 20 bp, Artificial Sequence
  Description of Artificial Sequence Synthetic primer
ccgcgactacagccactact

SEQ ID NO: 6 - DNA, 20 bp, Artificial Sequence
  Description of Artificial Sequence Synthetic primer
gagcctgttccgtctcaaac

SEQ ID NO: 7 - DNA, 20 bp, Artificial Sequence
  Description of Artificial Sequence Synthetic primer
atgttcgtcatgggtgtgaa

SEQ ID NO: 8 - DNA, 20 bp, Artificial Sequence
  Description of Artificial Sequence Synthetic primer
gtcttctgggtggcagtgat

SEQ ID NO: 9 - DNA, 20 bp, Artificial Sequence
  Description of Artificial Sequence Synthetic primer
gcctggtgctcctctatcta

SEQ ID NO: 10 - DNA, 22 bp, Artificial Sequence
  Description of Artificial Sequence Synthetic primer
ggctgttgaccatcataaaagc

(19) United States
(12) Patent Application Publication
(10) Pub. No.: US 2008/0248570 A1
(43) Pub. Date: Oct. 9, 2008

(54) Complexes of hyaluronans, other matrix components, hormones and growth factors for maintenance, expansion and/or differentiation of cells

(75) Inventors: William S. Turner, Chapel Hill, NC (US); Lola M. Reid, Chapel Hill, NC (US)

Correspondence Address: FOLEY AND LARDNER LLP;SUITE 500, 3000 K STREET NW, WASHINGTON, DC, 20007, US

(73) Assignee: University of North Carolina at Chapel Hill

(21) Appl. No.: 12/073,420

(22) Filed: Mar. 5, 2008

Related U.S. Application Data
(60) Provisional application No. 60/893,277, filed on Mar. 6, 2007.

(51) Int. Cl. C12N 5/06; C12M 1/00

(52) U.S. Cl. 435/377; 435/375; 435/289.1; 435/305.1; 435/325

(57) ABSTRACT

A method is provided of propagating hepatic cells including hepatic progenitors ex vivo on or in hyaluronans with or without other extracellular matrix components (such as collagens, basal adhesion molecules, proteoglycans or their glycosaminoglycans) and with or without hormones and/or growth factors. Compositions comprising the matrix are also disclosed. Also, the complex can be used for ex vivo tissue engineering or can be used as a scaffold for grafts of cells to be transplanted in vivo.

### CROSS-REFERENCE TO RELATED PATENT APPLICATIONS

This application claims priority from Provisional Application U.S. Application 60/893,277, filed Mar. 6, 2007, incorporated herein by reference in its entirety.

### FIELD OF THE INVENTION

The present invention relates generally to the maintenance, expansion and/or differentiation of cells such as liver cells, including hepatic progenitor cells. More particularly, the present invention relates to complexes of hyaluronans with other extracellular matrix components, hormones, and growth factors and used as scaffolds for maintenance, expansion and differentiation of cells, including progenitor subpopulations such as hepatic stem cells, hepatoblasts, committed progenitors and their progeny.

### BACKGROUND OF THE INVENTION

Maintenance of cells ex vivo is dependent on the use of nutrients, substrata of specific extracellular matrix components, and mixtures of soluble signals that include hormones and growth factors. Distinct defined mixtures of nutrients, matrix components and soluble signals elicit survival, expansion and differentiation of cells. Moreover, the composition of the defined mixtures is lineage dependent with specific compositions required for stem cells versus intermediates in the lineage versus mature cells. The mixtures complexed with hyaluronans offer a native, 3-dimensional (3-D) signaling scaffold, with an extent of solidity regulated by forms of cross-linking in addition to base matrix molecules, and all offer considerable advantages for tissue engineering ex vivo and for forms of grafts for cells to be reintroduced to animals (or people) in vivo. Such complexes are useful also for stem cells, for example, hepatic stem cells and their progeny (e.g., hepatoblasts and committed progenitors), that can be established in a complex comprised of a defined mixture of components to elicit dramatic 3-D expansion or can be seeded into ones that will drive 3-D differentiation. Stem cells are desirable candidates for cell-based therapies, including bioartificial livers or cell transplantation. This technology should facilitate such therapies especially for cells of solid organs in which grafting methods are likely to be especially important for the reintroduction of cells in vivo.

There is a need for conditions under which to achieve significant expansion of stem cells. This is dictated by the small numbers of the stem cells that can be isolated from normal tissues. By contrast, tissue engineering ex vivo or clinical programs of cell therapies can require very large numbers of cells to achieve desired endpoints. Therefore, technologies that permit self-renewal and/or extensive proliferation of stem cells to be followed by differentiation are greatly desired.

### SUMMARY OF THE INVENTION

In one embodiment, the present invention provides a method of maintaining, propagating and/or differentiating liver cells, including progenitors, ex vivo comprising: (a) providing a suspension of cells such as hepatic progenitor cells; and (b) culturing the cells in serum-free culture medium and on a complex of hyaluronans with or without other extracellular matrix components and with or without hormones or growth factors and in which the precise mixture of matrix components and hormones/growth factors facilitates 1) maintenance; 2) self-replication (also called self-renewal), 3) expansion (not involving self-renewal) and/or 3) differentiation of a population of cells that can be either progenitors or mature cells. The progenitors may be stem cells (e.g. hepatic stem cells), transit amplifying cells (e.g. hepatoblasts, candidate transit amplifying cells of liver), and/or committed (unipotent) progenitors (e.g. committed hepatocytic or biliary progenitors).

The extracellular matrix may further consist of hyaluronans complexed with collagens (such as a type I, III, IV or V collagen), basal adhesion molecules (such as laminins or fibronectins), proteoglycans or their glycosaminoglycan chains (such as heparin proteoglycan or heparins), and/or hormones (e.g. insulin) or growth factors (such as epidermal growth factor). In some embodiments the hyaluronans are chemically cross-linked, for example, through aldehyde bridges or disulfide bridges.

The cells of the invention are obtained from fetal, neonatal, pediatric or adult tissue. The serum-free culture medium can comprise insulin, transferrin, other hormones (e.g. tri-iodothyronine, growth hormone, glucagon, hydrocortisone), trace elements (e.g. zinc, copper, selenium), growth factors (e.g. epidermal growth factor or EGF, fibroblast growth factor or FGF, leukemia inhibitory factor or LIF) or a mixture; and in some embodiments may consist essentially of insulin, transferrin, lipids, and trace elements or essentially of insulin, transferrin, and lipids. Further, the calcium concentration in the media for epithelia can vary from that appropriate for expansion (<0.5 mM) to that for differentiation (>0.5 mM). Finally, the serum-free culture medium may be free of any growth factors or hormones other than insulin and transferrin.

Furthermore, the hyaluronan complexes of the instant invention may have application for ex vivo tissue engineering. For example, the complexes can be used as a scaffold for grafts for transplantation of cells in vivo.

In another embodiment of the invention, a method of propagating stem cells (e.g. hepatic stem cells) or transit amplifying cells (e.g. hepatoblasts) or a mixture of them ex vivo is provided comprising: (a) providing cells; and (b) culturing the cells in serum-free culture medium and on/in hyaluronans complexed with other extracellular matrix components and/or hormones or growth factors to propagate a population of progenitor cells without inducing their differentiation into committed progenitors. The lineage stage of the cells can be defined antigenically permitting recognition of self-renewal versus expansion with differentiation. For example, the hepatic stem cells can be defined as EpCAM+, NCAM+, Albumin +, CK19+, claudin 3+ AFP− and the liver's probable transit amplifying cells, hepatoblasts, are EpCAM+, ICAM-1+, Albumin+, AFP+, CK19+ and claudin 3−.

The extracellular matrix may further comprise hyaluronans complexed with one or more collagens, one or more basal adhesion molecules, one or more proteoglycans (or its/their glycosaminoglycan chains) and one or more hormone(s) or growth factor(s) or a mixture thereof. Further, in some embodiments the hyaluronans are chemically cross-linked, for example, through aldehyde bridges or disulfide bridges.

In yet another embodiment of the present invention, a composition is provided comprising a cell culture of isolated cells, serum-free culture medium, and hyaluronans complexed with or without other components. The extracellular matrix components further comprise any of a number of collagens, of basal adhesion molecules and/or proteoglycans or their glycosaminoglycan chains. As well, in some embodiments the hyaluronans are chemically cross-linked, for example, through aldehyde bridges or disulfide bridges.

In another embodiment, the hyaluronan complex is seeded with a mixture of epithelial cells (e. hepatic parenchymal cells) and certain mesenchymal cells (e.g. endothelia) and used as a graft for transplantation of the cells in vivo.

### BRIEF DESCRIPTION OF THE DRAWINGS

The patent or application file contains multiple figures executed in color. Copies of this patent or patent application publication with color figures will be provided by the Office upon request and with payment of the necessary fee.

FIG. 1 is an image showing hyaluronan receptors on hepatic progenitors. FIG. 1A shows hyaluaronan receptors on human hepatic progenitors in association with mesenchymal companion cells on culture plastic and stained for the hyaluronan receptors CD44 (Green) and Dapi (Blue). (10×). FIG. 1B-1D are images of freshly isolated hepatic progenitors showing receptors for CD44 (Green) and AFP (Red). (60×) Panels represented by B. CD44 C. AFP D. Overlay. FIG. 1E is a contrast image of hyaluronan receptor expression on an hepatic stem cell colony in comparison with the associated mesenchymal companion cells. Plates were stained with Bodipy conjugated hyaluronan. (4×). FIG. 1F-1I are composite images showing the varied cell types present on cultured plastic. A colony of human hepatic stem cells were stained for DNA (Dapi-Blue) or EpCAM (Green). Hepatic stellate cells expressing desmin are shown in red. (40× oil) Panels represented by A. DAPI B. EpCAM C. Desmin D. Overlay.

FIG. 2 shows the viability of cells grown within hyaluronan hydrogels. FIGS. 2A and 2B are phase contrast images of hyaluronan hydrogels seeded with human hepatoblasts and cultured for 20 days. (20×). 2C shows an aggregate (spheroid) of human hepatic progenitors cultured in hyaluronan hydrogels for 11 days and then dyed with Lysotracker (green; 488 nm) and Mitotracker (red; 543 nm) to indicate cell viability. The image shown is a confocal section of the spheroid at 40×/1.3 Oil DIC; scaling 0.06 μm×0.06 μm. 2D is a confocal sectioning through a spheroid showing viability of cells within the core of a spheroid within a hyaluronan hydrogel at day 11 of culture. Starting in frame 1 and ending in frame 6, the images “slice” through the spheroid showing live cells within the center. Stack Size: 1024×1024×45, 921.4 μm×921.4 μm×132.0 μm. Scaling: 0.9 μm×0.9 μm×3.0 μm. Objective Plan-Neofluar 10×/0.3 Wavelength: 543 nm. (Zeiss 510)

FIG. 3 shows certain antigenic expression of human hepatoblasts cultured in hyaluronan hydrogels. Aggregates of human hepatoblasts cultured in hyaluronan hydrogels were stained for various markers. All photographs were taken on a Zeiss 510, the Leica and Olympus FlowView confocal microscopes. FIG. 3A shows cytokeratin 19 (CK19) expression. Wavelength 488 nm. A 40× objective/1.3 Oil DIC Scaling 0.11 μm×0.11 μm was used. FIG. 3B is a phase micrograph of a spheroid of hepatic progenitors within the hyaluronan hydrogel using a 40× objective/1.3 Oil DIC. 3C is an overlay image of 3A and 3B. 3D shows albumin expression in the same culture of spheroids of cells as in 3B. Objective: Plan-Neofluar 40×/1.3 Oil DIC. Wavelength 543 nm. Stack size: 230.3 μm×230.3 μm. Scaling 0.22 μm×0.22 μm. Albumin expression is shown in red in human hepatoblasts within a hyaluronan hydrogel. FIG. 3E is a phase micrograph of hepatoblasts within a hydrogel. FIG. 3F is an overlay image of 3D and 3E. FIG. 3G shows cytokeratin (CK) 8 and 18 expression (green; Alexa 488 ). Nuclei were stained with Dapi (Blue). The hyaluronan hydrogel does not stain and appears as “wavy” images in the background. Use of a 60× Oil Immersion lense (Leica). FIG. 3H shows the expression of I-CAM/1 (Alexa 488; green) in cells within the spheroid of cells within a hyaluronan hydrogel. The nuclei are stained with DAPI (Blue). 60× Oil Immersion (Leica). FIG. 3I-3L show the expression of EpCAM, AFP, and albumin in cells maintained in hydrogel cultures. 20× with 6× zoom. (Olympus FV500). FIG. 3I. DIC (Black and White). FIG. 3J. EpCAM (Green). FIG. 3K. AFP (Red), 20× with 6× zoom. FIG. 3L. Albumin (Yellow). 20× with 6× zoom.

FIG. 4 shows evidence of the synthesis of albumin and urea by hepatoblasts cultured in hyaluronan (HA) hydrogels. FIG. 4A shows albumin production in cells in HA gels as compared to cells on plastic substrata determined over a course of 30 days in culture. The normalized albumin production of hepatic progenitors cells plated into HA hydrogels (open color coded circles) modulate in the collected culture and can be seen with a peak albumin production falling post days 8 and 9 (yellow color coded). The albumin data for the plastic (closed-filled circles) is shown under the data for the hydrogel conditions with all points falling beneath the lowest concentration detected for the hydrogels. No data line is fit for albumin production. FIG. 4B shows urea production in cells in HA gels versus on other substrata. The normalized mg/dl urea produced by hepatic progenitors in the hyaluronan hydrogels (upside down-open triangle) is compared to plastic (closed circles), collagen I gels (open circles) or a sandwich of collagen gels (filled triangles) cultures. Point to point curves are added to make day to day following of the graphed points easier.

FIG. 5 shows RNA expression of CK19, Albumin, and AFP (normalized to GAPDH). RNA encoding CK 19 (A), albumin (B), and AFP (C) was isolated from cultures of freshly isolated hepatoblasts, hepatic stem cells and hepatic progenitors cultured in the HA hydrogels. All values are normalized to the housekeeping gene, GAPDH and are expressed as the number of strands present per 30 ng of total RNA for the sample.

FIG. 6 shows hepatic stem cell colonies that were picked from plastic and transferred or passaged to the surface of a hyaluronan hydrogel cross-linked with disulfide bridges with or without associated collagen.

A. Hyaluronan hydrogel

B. Hyaluronan hydrogel with type I collagen

C. Hylauronan hydrogel with type III collagen

D. Hylauronan hydrogel with type IV collagen.

FIG. 7 shows hepatic stem cell colonies that were picked from cultures on tissue culture plastic and transferred to the surface of a hyaluronan hydrogel cross-linked with disulfide bridges and complexed with:

A. Laminin B. Laminin mixed with type I collagen

FIG. 8 shows hepatic stem cells embedded in an hyaluronan hydrogel cross-linked by disulfide bridges. Note that the cells are forming aggregates or spheroids throughout the hydrogels.

A. Hyaluronan hydrogels with embedded hepatic stem cells. 4×

B Hyaluronan hydrogels with embedded hepatic stem cells. 20×

### DETAILED DESCRIPTION OF THE INVENTION

In vivo, liver cells interact with both soluble factors (e.g., nutrients, gases, growth factors) and insoluble factors, such as the extracellular matrix components. Interactions with these factors—especially cell-to-cell interactions, availability of growth factors, and the presence or absence of specific extracellular matrix components found in mature liver tissue—have been studied. However, less studied have been the effects of matrix chemistries found predominantly in embryonic and fetal tissues.

Hyaluronans (HAs) are glycosaminoglycans (GAGs) consisting of a disaccharide unit linked with β-1-4, β-1-3 bonds between N-acetyl-D-glucosamine and glucuronic acid moieties, respectively. HAs contribute to matrix structure stabilization and integrity, water and protein homeostasis, tissue protection, separation and lubrication, facilitation of cell movement/migration, transport regulation (including steric exclusion), anchoring of hormones as a reservoir and integration of the immune inflammation response.

HAs are found in significant amounts in embryonic tissues and in adult tissues undergoing cellular expansion and proliferation, wound repair, and regeneration. In the liver, HAs are present in the matrix of embryonic and fetal tissues and near the presumptive stem cell compartment, the Canals of Hering, located in zone 1 of adult livers. However, HAs are not believed to be in association with the mature parenchymal cells. Therefore, the present inventors surmised that HAs could be candidate matrix components as 3-D scaffolds for ex vivo cultures of cells, especially progenitors, or as scaffolds for grafts for reintroduction of cells into hosts.

Hyaluronans have high turnover rates in vivo and yield scaffolds that are fragile and unstable, affecting their ability to be used in practical ways needed for ex vivo cultures, for tissue engineering, in bioreactor systems or in grafts for transplantation. Therefore, the HA scaffolds of the present invention are “stabilized” by chemical cross-linking. In some embodiments, the HAs are cross-linked through aldehyde bridges and in other embodiments the HAs are cross-linked via disulfide bridges.

The present inventors tested the biological effects of hyaluronans chemically modified through cross-linking, which rendered the HA hydrogel scaffolds insoluble in water, and yet maintained properties expected to be essential for their biological functions. Human hepatoblasts seeded into the HA hydrogels were found to retain their viability and their ability to divide for over 4 weeks, more than 3 times longer than possible with cells on culture plastic. It was discovered, surprisingly, that the cells seeded into pure hyaluronans (not complexed with other components) and with a medium, Kubota's Medium, designed for stem/progenitor cells and comprised only of basal medium, insulin, transferrin/fe, lipids, and two trace elements (selenium, zinc), remained stable (i.e., did not differentiate) and remained as stem cells or as very early stage hepatoblasts throughout the culture period. Although other culture conditions are permissive for survival and self-replication of hepatic stem cells (e.g. type III collagen and Kubota's Medium), hyaluronans have been the first culture condition identified that facilitates survival and self-replication of both stem cells and of hepatoblasts and the first that permits maintenance and self-replication in a 3-dimensional format. In monolayer formats, hepatoblasts require various feeders for survival and demonstrate limited expansion potential on the feeders identified to date; indeed, hepatoblasts have been found to self-replicate only on hyalurnonans and under no other conditions tested.

Livers are comprised of a mixture of hemopoietic, mesenchymal, and hepatic progenitor cells. The hepatic progenitor subpopulations in livers consist of two pluripotent cell populations—hepatic stem cells and hepatoblasts—and two unipotent populations—committed hepatocytic progenitors and committed biliary progenitors.

The hepatic stem cells and hepatoblasts have extensive overlap in their phenotype; expressing albumin, epithelial-specific cytokeratins (CK) 8 and 18, a biliary-specific cytokeratin CK19, epithelial cell adhesion molecule EpCAM (CD326 or HEA125), CD133/1 (prominin), telomerase, Sonic and Indian hedgehog, and being negative for hemopoietic (CD45, CD34, CD38, CD14, and glycophorin A), endothelial (CD31, VEGFr or KDR, Van Willebrand factor), and other mesenchymal (CD146, desmin, a-smooth muscle actin or SMA) markers. They are distinguishable in that hepatic stem cells express NCAM and claudin 3, whereas hepatoblasts express ICAM-1 (CD54), alpha-fetoprotein (AFP), and fetal P450s (e.g. P450A7) (see Table 1). In vivo, the pluripotent hepatic progenitor cells give rise to the hepatocytic and biliary lineages between the 11th and 13th weeks of gestation.

TABLE 1
Lineage-dependent Markers of Parenchymal Cell Lineages:

| | | | Adult Hepatocytes |
|---|---|---|---|
| | Hepatic Stem | | (Adult Biliary |
| | Cells | Hepatoblasts | Epithelia) |
| EpCAM | +++ | ++ | −− |
| | | (on some but | (++) |
| | | not all) | |
| AFP | −−− | +++ | −− |
| | | | (−−) |
| Albumin | + | ++ | +++ |
| | | | (−−) |
| CK19 | +++ | ++ | −− |
| | | | (++) |
| Claudin 3 | +++ | − | − |
| | | | (+) |
| Telomerase | +++ | +++ | ++ |
| | | | (n.t.) |
| Sonic and | +++ | ++ | −− |
| Indian | | | (−−) |
| Hedgehog | | | |
| I-CAM1 | −−− | +++ | ++ |
| | | | (+) |
| N-CAM | +++ | −−− | −− |
| | | | (−−) |
| MDR3 | − | − | −− |
| | | | (+++) |
| P450-3A4 | −−− | −−− | +++ |
| | | | (−−) |

EpCAM = epithelial cell adhesion molecule; CK19 = cytokeratin 19, a biliary specific cytokeratin; I-CAM = intercellular adhesion molecule; NCAM = neuronal cell adhesion molecule; MDR3 = multidrug resistance gene isoform 3 (involved in bile transport) P450-C3A4 = cytochrome P450 3A4; Claudin 3 = tight junction protein (isoform 3), n.t = not tested.

The present invention provides a method of maintaining, expanding and/or differentiating cells, including progenitors, over long periods of time. The cells can be established under survival, expansion or differentiation conditions depending on the exact mixture of components complexed to the hyaluronans and to the precise composition of the serum-free, defined medium. In one embodiment, hepatic progenitors, hepatoblasts or hepatic stem cells, are obtained from human livers and propagated on/in hyaluronan hydrogels with “Hiroshi Kubota's Medium,” (HK) being a serum-free basal medium with low or no copper, low calcium (<0.5 mM), and supplemented only with insulin, transferrin/fe, lipids (high density lipoprotein and free fatty acids bound onto purified albumin), and certain trace elements (zinc, selenium). This method also provides a means for stable propagation of cells having a phenotype, which under these conditions, is intermediate between that of stem cells and hepatoblasts. In this way, HA hydrogels, in combination with a serum-free medium tailored for hepatic progenitors (e.g., HK medium) can provide a suitable three-dimensional scaffolding for human hepatic progenitors, in this case for stem cells and early stages of hepatoblasts. The hydrogel plus the medium also enables the maintenance of cells as early stage hepatoblasts in terms of viability, with proliferative capacity, with phenotypic stability through prolonged culture periods, and with minimal, if any, lineage restriction towards either biliary or hepatocytic fates.

Without being held to or bound by theory, it is presently believed that HAs that are aldehyde cross-linked via, e.g., the carboxyl groups of HA, are poorly modified by enzymatic activity from cells (e.g. angioblasts or endothelia) that are companion cells to the hepatic stem cells, and result in slowed growth of the hepatic progenitors on the HAs. Extracellular matrix turnover, including that of the hyaluronans, typically is accomplished in vivo by enzymatic digestion by cells, an intrinsic process in the expansion and establishment of cells to form a tissue or organ. Hence, it is presently believed that progenitors ex vivo require the ability to digest the HAs in order to expand. The stiffness of the HA scaffold also could affect the maturation of the cells as could the large fluidic volume contained within the hydrogel. Therefore, the physicochemical properties (such as flexibility and cross-linking density) of the HA hydrogel should be modulated to optimize cell expansion processes.

### EXAMPLES

### Sourcing of Human Livers

Fetal Livers. Liver tissue was provided by an accredited agency (Advanced Biological Resources, San Francisco, Calif.) from fetuses between 18-22 weeks gestational age obtained by elective terminations of pregnancy. The research protocol was reviewed and approved by the IRB for Human Research Studies at the UNC.

Postnatal Livers. Intact livers from cadaveric neonatal, pediatric and adult donors were obtained through organ donation programs via UNOS. Those used for these studies were considered normal with no evidence of disease processes. Informed consent was obtained from next of kin for use of the livers for research purposes, protocols received Institutional Review Board approval, and processing was compliant with Good Manufacturing Practice.

### Cell Isolation

Fetal livers. The methods for processing human fetal liver tissues have been previously reported, for example, in Schmelzer E. et al. 2006 (Stem Cells). All processing and cell enrichment procedures were conducted in a cell wash buffer composed of a basal medium (RPMI 1640) supplemented with 0.1% bovine serum albumin (BSA Fraction V, 0.1%, Sigma, St. Louis, Mo.), insulin and iron saturated transferrin both at 5 ug/ml (Sigma St Louis Mo.) trace elements (selenious acid, 300 pM and ZnSO4, 50 pM), and antibiotics (AAS, Gibco BRL/Invitrogen Corporation, Carlsbad, Calif.). Liver tissue was subdivided into 3 mL fragments (total volume ranged from 2-12 mL) for digestion in 25 mL of cell wash buffer containing type IV collagenase and deoxyribonuclease (Sigma Chemical Co. St Louis, both at 6 mg per mL) at 32 EC with frequent agitation for 15-20 minutes. This resulted in a homogeneous suspension of cell aggregates that were passed through a 40 gauge mesh and spun at 1200 RPM for five minutes before resuspension in cell wash solution. Erythrocytes were eliminated by either slow speed centrifugation or by treating suspensions with anti-human red blood cell (RBC) antibodies (Rockland, #109-4139) (1:5000 dilution) for 15 min followed by LowTox Guinea Pig complement (Cedarlane Labs, # CL4051) (1:3000 dilution) for 10 min both at 37° C. Estimated cell viability by trypan blue exclusion was routinely higher than 95%. See supplemental data for further details.

Postnatal livers. The livers were perfused through the portal vein and hepatic artery for 15 min with EGTA-containing buffer and then with 600 mg/L collagenase (Sigma) for 30 min at 34° C. The organ was then mechanical dissociated in either collection buffer; the cell suspension passed through filters of pore size 1,000, 500, and 150 microns; the single cells collected and then live cells fractionated from dead cells and debris using density gradient centrifugation (500×g for 15 min at room temperature) in Optiprep-supplemented buffer in a Cobe 2991 cell washer. The resulting hepatic cell band residing at the interface between the OptiPrep/cell solution and the RPMI-1640 without phenol red was collected.

In other experiments, viability was assessed in cultures using one of several vital dyes: Lysotracker Green, Mitotracker Red, and Lysotracker Red (Molecular Probes). Preferably, a dye was chosen based on its contrast to other fluoroprobes when co-staining. The vital dyes were incubated for 30 minutes in HK media and at the following concentrations: 75 nM Lysotracker Green, 75 nM Lysotracker Red, and 250 nM Mitotracker Red.

### Tissue Culture Plastic

Suspensions of the human hepatic progenitors, enriched for hepatoblasts, were seeded onto plastic with a 2.5% Fetal Bovine Serum (FBS) addition to the HK medium. After 16 hours of incubation at 37° C. with 5% CO₂, the media was replaced with serum free HK media for the remainder of the study. Cells on plastic were cultured with media changes every 3 days, until the end of the experiment. Cells that did not attach within the first 16 hrs of culture were aspirated at times of media change. At the experiments end, the cells were fixed with 4% paraformaldehyde added to the plate after aspiration of the HK media.

### Human Hepatic Stem Cells and Hepatoblasts Have Receptors for Hyaluronans

Cells were stained for immunofluorescence using primary antibodies directly labeled with the relevant fluoroprobe or two-step staining with primary antibodies followed by secondary antibody coupled to the fluoroprobe (see Table 2, below). Prior to staining, approximately 1 ml of Phosphate Buffer Solution (PBS) was placed on the site of interest to wash any debris away. Goat serum (10% in PBS solution) was added for 1 hour to block non-specific binding sites within the tissue. Blocking was removed and the site washed with 1× PBS. Monoclonal antibody was added and incubated overnight. After an overnight (e.g., 18 hour) incubation at 4° C., the primary monoclonal antibody solution was removed, and the sample was washed three times with 1× PBS for 10 minutes each time. Secondary antibody (Alexa 488 or Alexa 594, Molecular Probes) was added at a dilution of 1:750 or 1:1000. The sample was covered from light exposure and left for 1 hour incubation at room temperature. Samples were washed 3 times with 1× PBS and prepared with cover slips using either DPX mounting media (Electron Microscopy Sciences) for microscopy or Vector Shield containing DAPI mounting media (Vector Laboratories).

DAPI concentration was 1.5 μg/ml. Hepatic fetal stem cell colonies were fixed after 10 days in culture with 4% para-formaldehyde in PBS, and blocked for 1 hour at room temperature with 10% goat serum in PBS 0.1% Triton-X100. Primary antibodies rabbit IgG anti desmin (Abcam) and mouse IgG1 anti EpCAM (Labvision) were applied in blocking buffer for 1 hour at room temperature; secondary antibodies anti-rabbit AlexaFluor 568, anti-mouse IgG1 AlexaFluor 488 conjugated (Molecular Probes/Invitrogen), and DAPI (Sigma) for nuclei staining were applied in blocking buffer for 1 hour at room temperature. Fluorescence was analyzed using a Leica SP2 laser scanning confocal microscope controlled by Leica SP2 TCS software (Leica Microsystems).

For analysis of cytoplasmic antigens (e.g. albumin, AFP) coupled to a fluorochrome label, cells were imaged with a LeicaSP2 AOBS Upright Laser Scanning Confocal, a Zeiss 510 Meta Inverted Laser Scanning Confocal Microscope, and a Leica DMIRB Inverted Fluorescence/DIC Microscope—with B/W & Color digital cameras.

TABLE 2
Antibodies and Fluoroprobes

| | | |
|---|---|---|
| Reagent | Dilution | Source |

Primary Antibodies

| | | | |
|---|---|---|---|
| | | Isotype | |
| Cytokeratin 19 (CK19)-biliary | 1:500 | IgG | Amersham |
| specific cytokeratin | | | |
| Cytokeratins 8/18 (CK 8/18)- | 1:800 | IgG | [Zymed |
| epithelial-specific cytokeratins | | | |
| Hyaluronan receptor (CD44), a | 1:300 | IgG | Molecular Probes |
| hyaladherins | | | (Invitrogen) |
| Albumin | 1:800 | IgG | Sigma |
| Alpha-fetoprotein | 1:200 | IgG | Zymed |
| ICAM-1 (CD54) | 1:1000 | IgG | PharMingen |
| Desmine | 1:800 | IgG | AbCam |
| EpCAM | 1:800 | IgG | Molecular Probes |
| | | | (Invitrogen) |

Fluoroprobes

| | | | |
|---|---|---|---|
| | | Excitation/ | |
| | | Emission | |
| Alexa 647 (far red) | 1:500 | | Sigma |
| Alexa 594 (red) | 1:750 | 590/617 | Sigma |
| Alexa 488 (Green) | 1:1000 | 495/519 | Molecular Probes |
| DAPI (blue) | 1:1000 | 358/461 | Molecular Probes |
| HA-Bodipy Conjugate | 1:100 | 485/530 | Invitrogen |

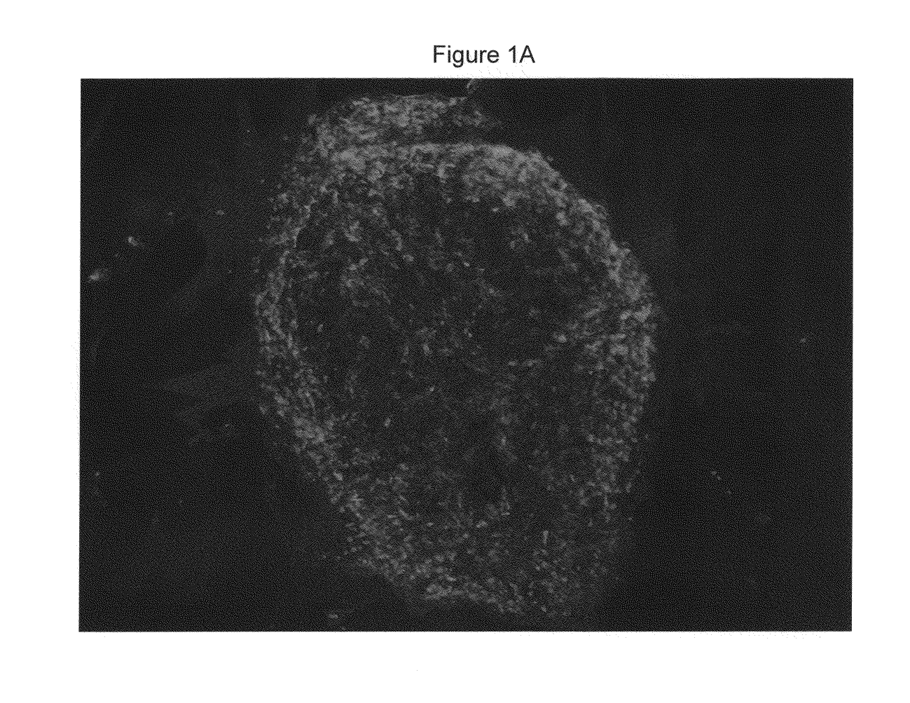

The results indicate that human hepatic stem cells and hepatoblasts are positive for hyaluronan receptors as evidenced by immunostaining of a tightly packed, 25 day-old colony of human hepatic stem cells with fluorescent antibodies to CD44 as shown in FIG. 1A. Freshly isolated hepatoblasts, which are AFP positive are also shown to be positive for the CD44 receptor in FIG. 1B-D. CD44, a cell surface glycoprotein, is indicated in green, which highlights a receptor for the HA attachment. The receptors cover nearly 100% of the cells in the stem cell colony in FIG. 1A, with individual cells containing varied amounts of the receptor seen as intense staining in some cells and lighter less intense staining of others. Individual cells are contrasted by use of DAPI staining (blue) of their nuclei.

As shown, the stainings imply that each human hepatic progenitor cell has HA attachment capabilities. In FIG. 1E, primary cultures of human hepatic progenitor cells, isolated from human fetal livers and cultured on plastic for 4 weeks, were imaged at 4× and are fluorescently stained for a HA-BODIPY conjugate. The hepatic progenitors express levels of receptors for HA at higher rates than other cells evident in the culture and that include stroma and endothelial cells. Hepatic progenitors, with heavy BODIPY staining due to uptake of the conjugated HA are located in the lower left quadrant.

Comparatively, fibroblasts and non-parenchymal cells shown respectively in the lower right and upper quadrants are less active in their HA mediated binding and uptake. Immunohistochemical staining of the nonparenchymal cells has been done utilizing markers defined by others to identify specific subpopulations. The mesenchymal cells comprise multiple subpopulations that include angioblasts (KDR+/CD133-1+/CD117+); mature endothelia, (CD31+); hepatic Stellate Cells (desmin+, alpha-smooth muscle actin+); hemopoietic cells (CD45+) including red blood cells (glycophorin A+). Representatives of these cellular subpopulations are those shown in FIGS. 1F-I (hepatic stellate cells positive for desmin expression located adjacent to EpCAM positive stem cells)

### Human Hepatic Progenitors are Viable and Expand 3-Dimensionally in HA Hydrogels

Hyaluronan (average MW: 1,500,000) was obtained from Kraeber GMBH and Co. (Waldhofstr, Germany). Adipic dihydrazide (ADH) and Ethyl-3-[3-dimethyl amino] propyl carbodiimide (EDCI) was purchased from Sigma-Aldrich (St. Louis, Mo). These, and other reagents disclosed herein, are available from multiple vendors, all of which supply reagent suitable for practice with the instant invention. Hyaluronan matrices configured for cell culture were prepared by aldehyde cross-linking using a method modified from previously published protocol. See, e.g., Vercruysse K P, et al., Synthesis and In Vitro Degradation of New Polyvalent Hydrazide Cross-Linked Hydrogels of Hyaluronic Acid. Bioconjugate Chemistry 1997; 8:686-694; and Kim A. et al., Characterization of DNA-hyaluronan matrix for sustained gene transfer. Journal of Controlled Release 2003; 90:81-95; the disclosures of which are incorporated herein in their entirety by reference.

Briefly, a 1% aqueous hyaluronan solution was prepared, measured and deposited in aluminum molds of proper sizes, snap frozen on dry ice and lyophilized to form solid, spongy wafers. The wafers were incubated in a 0.1% ADH solution (90% isopropanol/10% water) for 30 minutes to enable the complete penetration of the ADH solution. EDCI (120 mg) was added to the ADH solution and quickly dissolved upon agitation. Cross-linking of the partially hydrated HA spongy wafers was initiated by adding 1N HCl to the reagent mixture to adjust the pH to approximately 4.5.

The reaction was terminated by decanting the reagent mixture and replacing it with 100 ml of 90% isopropanol. The cross-linked HA matrices recovered were subsequently extracted with 100 ml of 90% isopropanol at least 5 times by incubating overnight. The HA matrices were then transferred to pure isopropanol to remove all residual water and air dried. The diameters of the cross-linked HA matrices were 0.7 or 3.5 cm, respectively. Upon re-hydration, the HA matrices readily absorbed water and formed highly porous HA spongy hydrogels. Prior to use in culture, HA hydrogels were sterilized by exposure to a Cesium source (JL Shepard Mark I Model 68 Cesium Irradiator—Department of Radiation Oncology, UNC) with a deliverable dosage of 40 Gray (40 Joule/kg), over a 10 minute period.

### Hepatoblast Cultures in Hyaluronan Hydrogels

HA hydrogels were placed into culture wells, either 6-well culture treated polystyrene, or for the smaller sized hydrogel matrices, chambered coverglass culturing slides (Lab-Tek-Nunc, Napersville, Ill.). Smaller hydrogels required no manipulation (priming) prior to inoculation with freshly isolated cells other than a pre-soak with HK media. The larger hydrogels benefited from slight manipulation to insure the removal of air bubbles from the hydrogels. In most cases, addition of 3 ml of HK media onto the hydrogel would trap air bubbles, which could be removed mechanically by slight compression-relaxation of the hydrogel, forcing air from the lateral sides.

After priming, suspensions of human hepatic progenitors, enriched for hepatoblasts, were seeded onto large HA hydrogels at 2×10⁶ cells/hydrogel in HK medium with 2.5% FBS and at 2×10⁵ cells per small hydrogel. After 16 hours initial incubation at 37° C. in a CO₂ incubator, the medium with FBS was replaced with serum-free HK. The working volume for a 6 well plate was 3 ml and for the 2-chambered wells was 2 ml. Cells were cultured for 4 weeks under these same conditions, with changes of the media every 2-3 days.

As discussed herein, HK media comprised of a serum-free basal medium (e.g., RPMI 1640, Gibco—Invitrogen) containing no copper, low calcium (<0.5 mM) and supplemented with insulin (5 μg/ml), transferrin/fe (5 μg/ml), high density lipoprotein (10 μg/ml), selenium (10-10 M), zinc (10-12 M) and 7.6 μE of a mixture of free fatty acids bound to purified albumin. The detailed methods for the preparation of this media have been published elsewhere, e.g., Kubota H, Reid L M. Clonogenic hepatoblasts, common precursors for hepatocytic and biliary lineages, are lacking classical major histocompatiblity complex class I antigen. Proceedings of the National Academy of Sciences (USA) 2000; 97:12132-12137, the disclosure of which is incorporated herein in its entirety by reference.

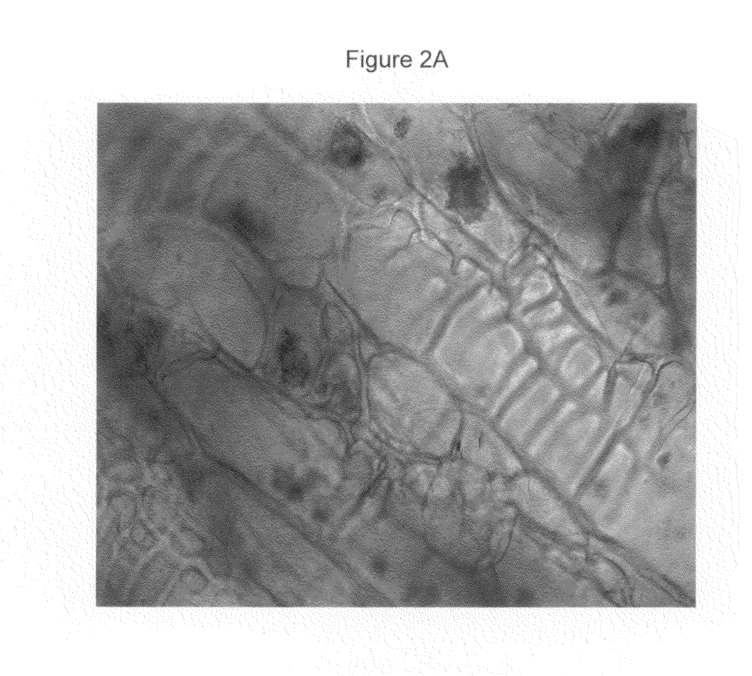

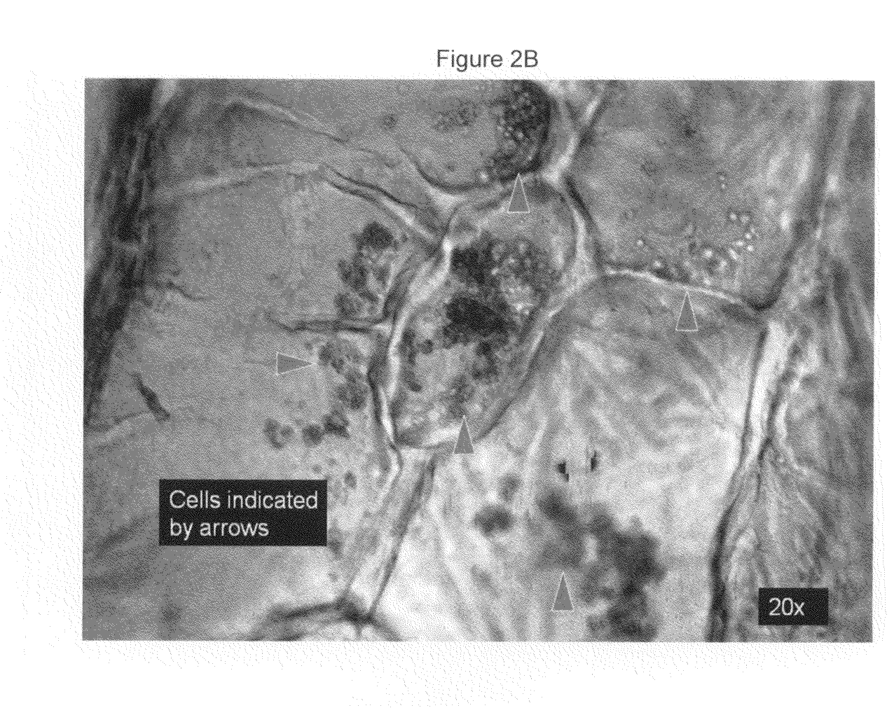

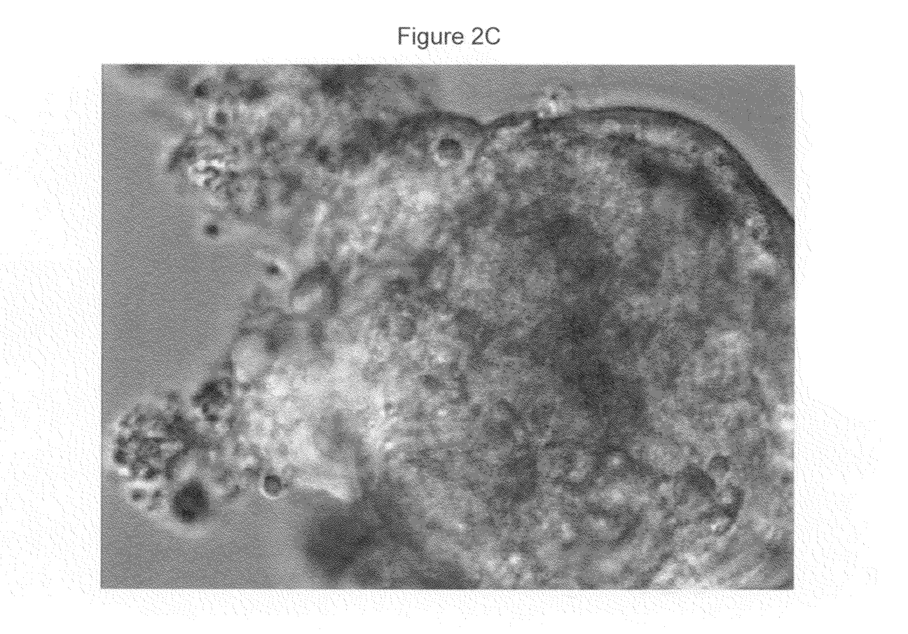

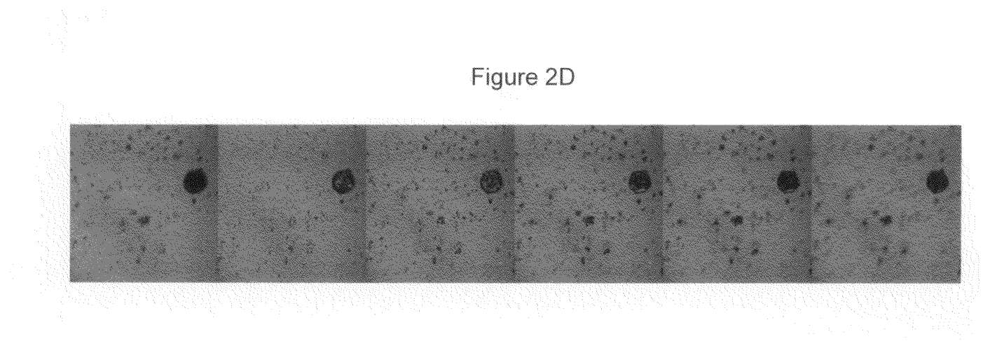

Cells isolated from freshly dissociated human fetal livers show an affinity for aggregation/expansion in the hydrogels. Single cells and aggregates with up to four cells/aggregate were initially seeded within the HA hydrogels. Cells aggregates, at the end of a 3 week culturing period, shown in FIGS. 2A, 2B, 2C and 2D show much larger cell aggregates. Sampled aggregates of FIG. 2B have cell counts ranging between 63 and 2595 cells per aggregate. FIGS. 2A and 2B illustrate visible aggregate spheroids within the HA hydrogel.

Furthermore, the aggregates in FIGS. 2C and 2D display cell viability with fluorescence capture of Mitotracker and Lysotracker activity, where the fluoroprobe is cleaved into a visible component after active uptake. FIG. 2D also represents a confocal plane that shows the aggregate spheroid is neither hollow nor necrotic within the interior (Mitotracker-red, stained) frames 2-5. DNA measurement shows a complete reversal of quantifiable cell DNA collected from the death of cells on plastic versus their expansion in the HA hydrogel with an average daily increase of about 2% over a 14 day incubation period.

### Hepatoblasts Survive Longer in Hyaluronan Hydrogels in Comparison to Those on Culture Plastic

Suspensions of the human hepatic progenitors, enriched for hepatoblasts, were seeded onto plastic with a 2.5% Fetal Bovine Serum (FBS) addition to the HK medium. After 16 hours of incubation at 37° C. with 5% CO₂, the media was replaced with serum-free HK media for the remainder of the study. Cells on plastic were cultured with media changes every 3 days, until the end of the experiment. Cells that did not attach within the first 16 hrs of culture were aspirated at times of media change. At the end of the experiment, cells were fixed with 4% paraformaldehyde.

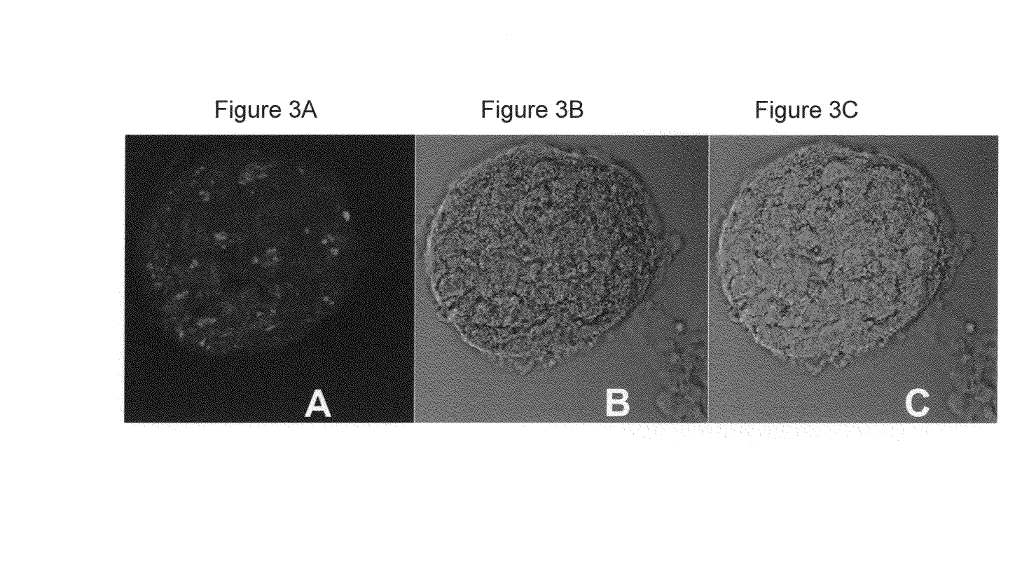

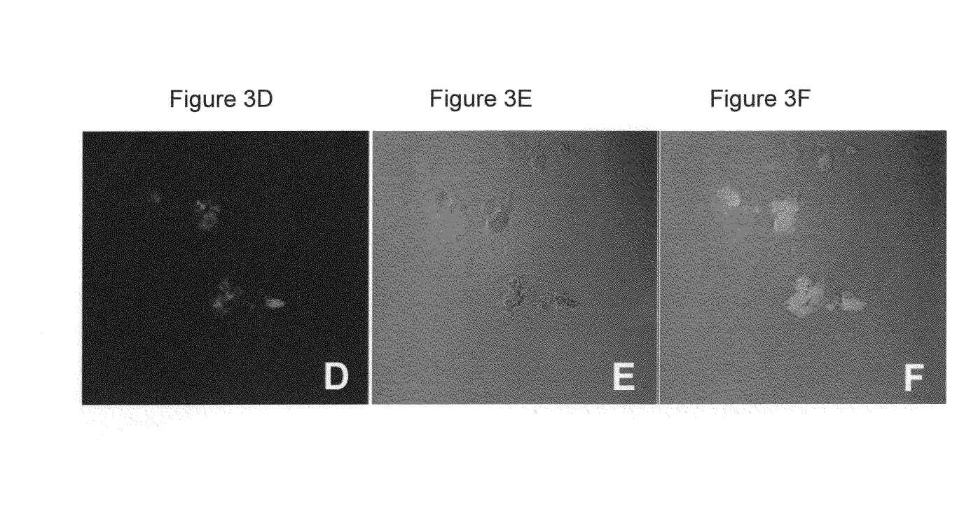

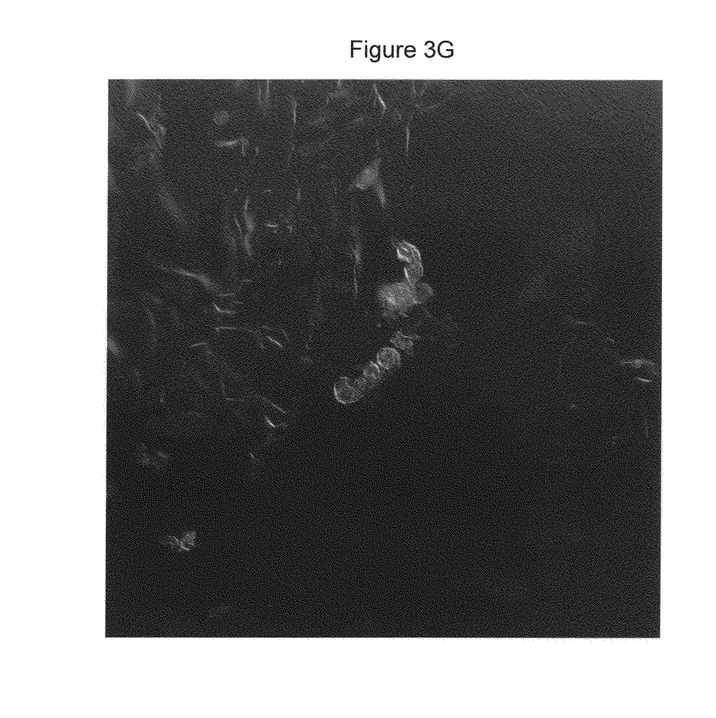

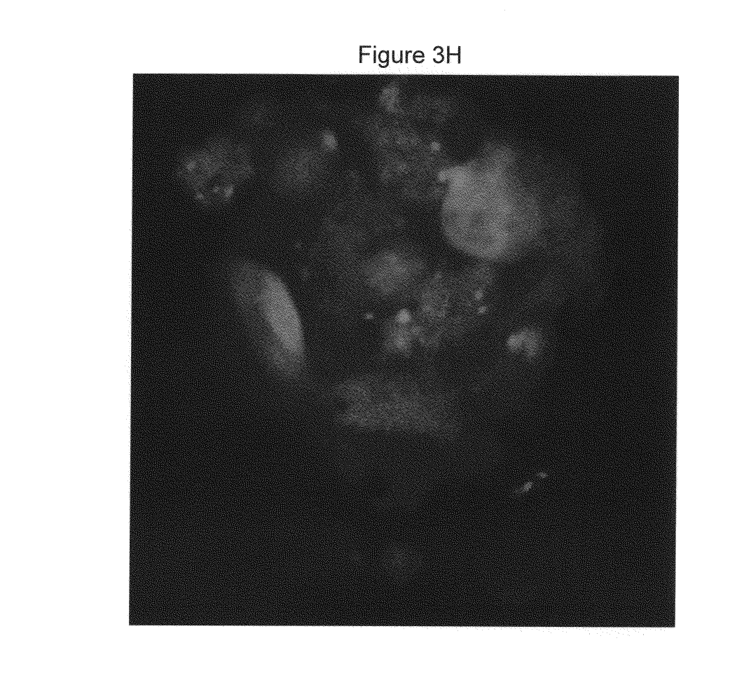

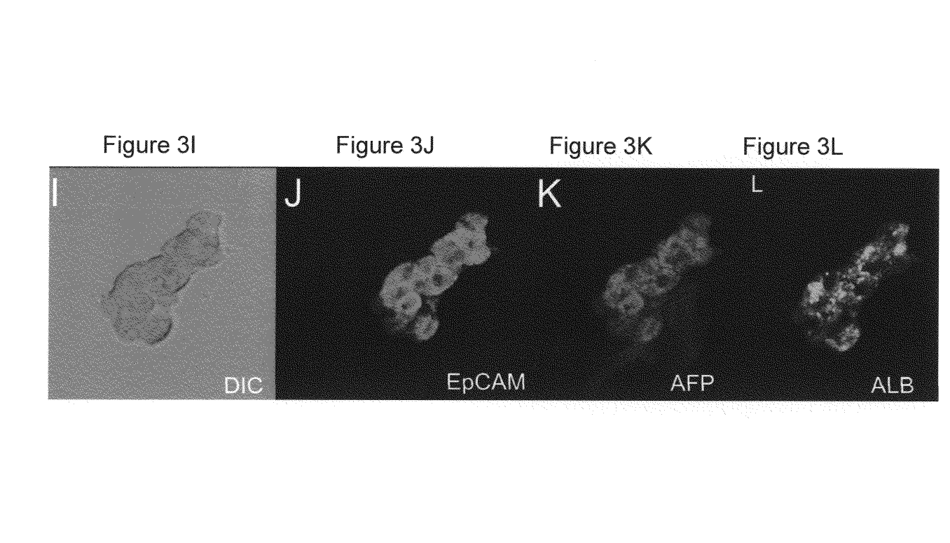

Cells in the hydrogel hydrogels and in the HK medium maintained a stable phenotype intermediate between that for hepatic stem cells and hepatoblasts throughout more than 4 weeks of culture and did not lineage restrict towards either biliary or hepatocytic fates. Representative data are shown by immunohistochemistry staining given in FIG. 3. The cells are hepatic parenchymal progenitors as evidenced by their co-expression of the biliary lineage marker, CK19 with albumin (FIGS. 3A-3F) and are epithelia as evidenced by their staining for CK8/18 (FIG. 3G). The I-CAM staining (FIG. 3H) found in the majority of the cells and the low levels of expression of AFP indicates the cells are held in a differentiated state close to that of hepatoblasts. Indeed, fully differentiated hepatoblasts would be expected to have very high levels of alpha-fetoprotein, a conclusion corroborated by biochemical assays for functions (see below). Finally, hepatoblasts are marked by the co-expression of three markers: EpCAM, AFP, and Albumin (FIG. 3i-l).

### Cells Maintain Phenotype of Early Stage Hepatoblasts for Longer than 4 Weeks in HA Hydrogels

Albumin production was measured by enzyme-linked immunosorbent assay (ELISA). The media supernatant was collected from control (plastic) cultures and the HA hydrogels once every day or every other day for the duration of a 4-week culture period. Media from the culture were frozen and stored at −20° C. until analyzed. Purified human albumin was used as the standard, and peroxidase-conjugated antibody was used as the fluoroprobe against albumin. Measurements were made with a Spectromax 250 multi-well plate reader (Molecular Devices, Sunnyvale, Calif.).

Similarly, urea production was analyzed using the urea nitrogen sensitivity assays, based on direct interaction of urea with diacetyl monoxime. Urea concentration was measured spectrophotometrically at 515-540 nm with a cytofluor Spectromax 250 multi-well plate reader. Albumin production of the hepatic progenitors cultured in HA hydrogels was compared to that of hepatic progenitors cultured on plastic over the course of 30 days of culture. The concentration of albumin (per volume) peaked between Days 7 and 10 for all cultures. Hepatoblasts lasted 7 to 10 days in cultures on plastic and reliably expressed significant levels of albumin. By contrast, the hepatic progenitors lasted for more than 4 weeks in the cultures in HA hydrogels.

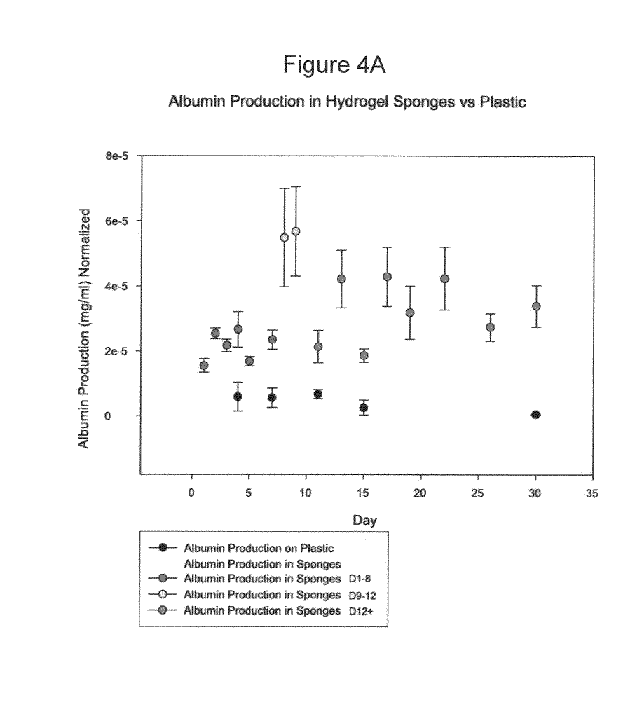

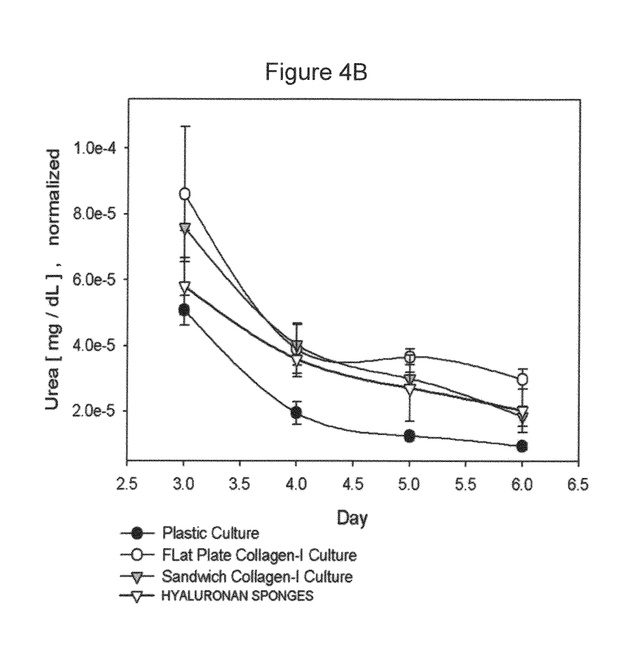

FIG. 4A is the normalized albumin production of hepatoblasts plated into HA hydrogels (Open Color Coded Circles). The albumin levels spike and fall between days 8 and 10, similar to that of cells plated onto culture plastic and on type I collagen substrata. The normalized amount of albumin is markedly higher, modulating about a trend nearing 4.0×10⁻⁵ mg/ml, whereas hepatoblasts cultured on plastic are well below the 2.5×10⁻⁵ mg/ml baseline. When the albumin data for the cells on plastic (Closed-Filled Circles) is plotted relative to that for cells in the hydrogels, the normalized data is consistently lower than the same cells cultured in the HA hydrogels.

Where collagen gels are utilized in this study, rat tail collagen type I is used. The collagen matrix has a density concentration of 1.5 mg/ml, unless specified otherwise. For flat plate cultures of this investigation, 0.4 ml of collagen-I is plated over the 35 mm diameter culture surface and incubated for 1 hour at 37° C. and 95% O₂-5% CO₂ to allow gelation. Then, 1 million viable hepatocytes are seeded onto the gelled layer using media supplemented with 10% FBS. Following 8 hours of cell incubation, the medium is removed and 0.5 ml of serum-free culture media is added to the top of the culture, and changed daily.

For sandwich culture studies of this investigation, the culture incorporates a 35 mm tissue culture dish. Briefly, 1 million viable cells were plated on a flat plate collagen matrix and allowed to attach for 8 hours in media supplemented with 10% FBS at 37° C. and 5% CO₂. The media is then removed and an additional 0.4 ml of collagen is applied to the top of the cells, followed by gelation for 1 hour at 37° C. Next 0.5 ml of serum free culture media was added to the top of the culture, and changed daily.

Urea production, a common function for mature hepatocytes, is represented graphically in FIG. 4B. The concentration of urea is given in mg/dl for this assay. Normalized mg/dl urea production by hepatoblasts in hyaluronan hydrogel hydrogels (upside down-open triangle) are compared to that from cells on plastic (Closed Circles), cells on monolayer collagen I cultures (Open Circle), and cells cultured between two layers of type I collagen (Hash filled triangles). Again, there is a decrease in production in all cultures with the HA hydrogels performing slightly better than plastic, and forming a slower falling decay.

Isolation of RNA cells cultured in HA hydrogels was done using TRizol isolation provided by Invitrogen. Hydrogels were removed from the culture plates and placed into 2 ml Eppendorf tubes, and spun at 12,000 rcf (11,953.34 g) on a microfuge at 4° C. Supernatant was removed by aspiration and 1 ml of TRIzol was added. In comparative plastic control cultures, where cells were adherent to the culture plates, TRIzol was added directly to the plates and then collected into tubes without spinning, but after aspiration of the media.

RNA was collected via phase separation with addition of 0.2 ml chloroform. After aqueous phase collection, RNA was precipitated via isopropyl alcohol, followed by a 70% ethanol wash. Final preparations of RNA were air-dried and resuspended in 100 ul of RNase free water. Quantification was done with a DU7400 Spectrophotometer (Becker).

DNA was isolated by addition of 0.3 ml of 100% ethanol to each tube of the remaining TRIzol. Tubes were incubated for 2 minutes at room temperature, and then centrifuged at 1000 g, 4° C., for 5 minutes. The phenol/ethanol aqueous phase was removed for further analysis of the protein. The DNA pellet was washed twice with sodium citrate solution, then with 75% ethanol, and centrifuged each time at 5000 g at 4° C. After a second ethanol spin, supernatant was removed by aspiration, and the sample was air dried for 15 minutes. The pellet was re-dissolved in 100 ul of 8 mM NaOH and buffered with 3.2 ul 1M Hepes (Mediatech) for a final pH of 7.0. The samples were spun at 12000 g for 10 minutes and the supernatant was transferred to a new tube. DNA quantification was done with the Beckman Photospectrometer.

### Gene Expression as Analyzed by Quantitative Real Time RT-PCR

Gene specific mRNAs were created as followed: total RNA from livers was extracted using the RNeasy kit (Qiagen, Valencia, Calif.) and reverse transcribed by Superscript II reverse transcriptase (Invitrogen) and oligo-dT(I12-18) primer. cDNA was used as the template in conventional PCR with gene specific primers (for sequences see Table 3, below) from which the forward primer possessed an 5′ overhang for T7-promotor sequence (5′gac tcg taa tac gac tca cta tag gg). This amplified gene specific DNA was used for in vitro transcription with T7-RNA polymerase (Promega), generating gene specific RNA (with an additional 5′ggg included by T7-RNA polymerase) used as standards in quantitative RT-PCR using gene specific primers without 5′ overhang; standard ranges were linear from 1 to 108 templates. Quantitative RT-PCR was done in the LightCycler instrument (Roche) using the LightCycler RNA Master SYBR Green I kit. RNA from samples was extracted using RNeasy mini kit (Qiagen).

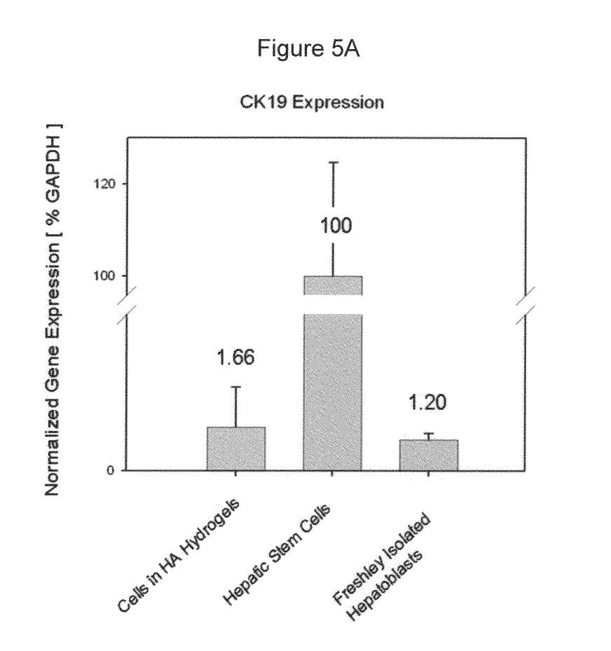

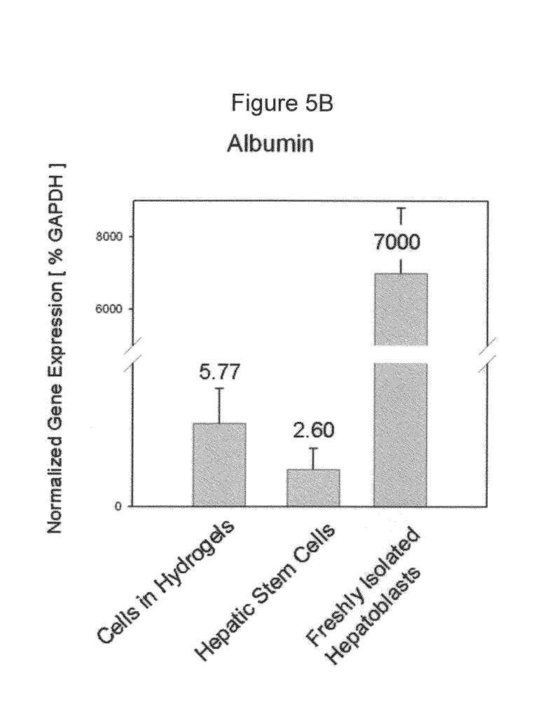

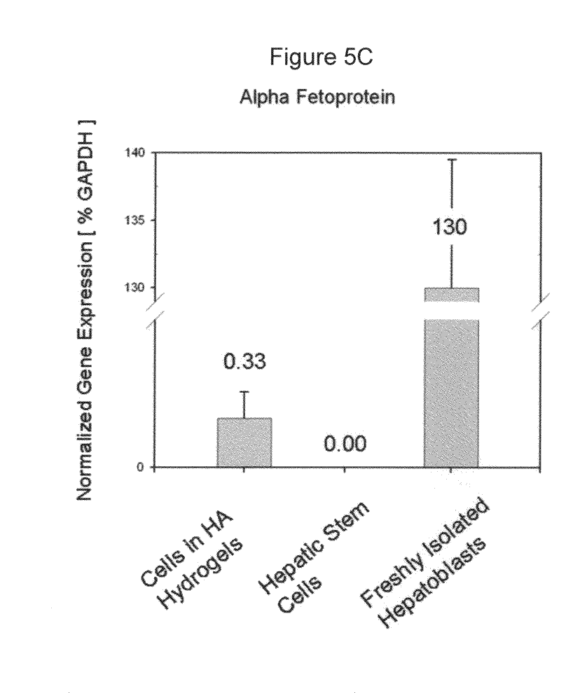

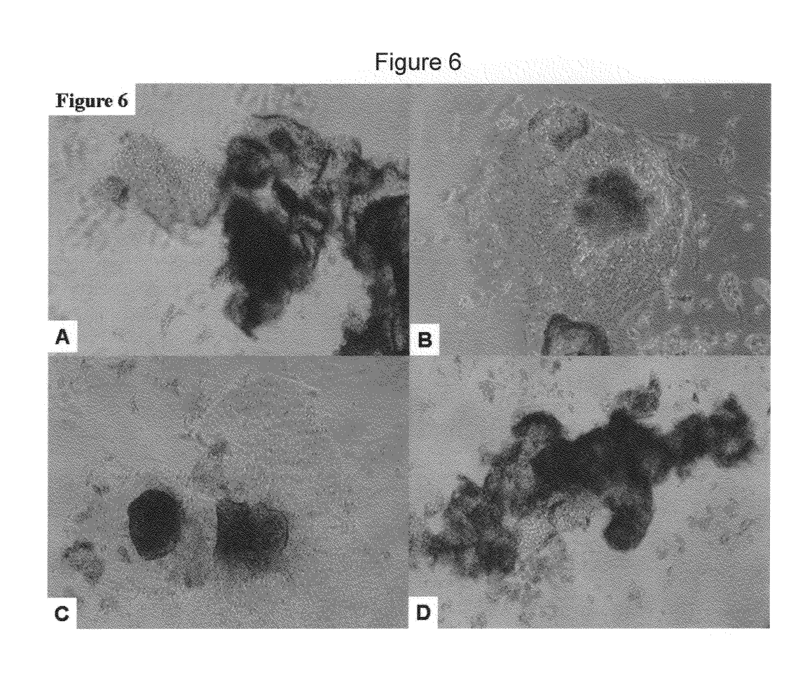

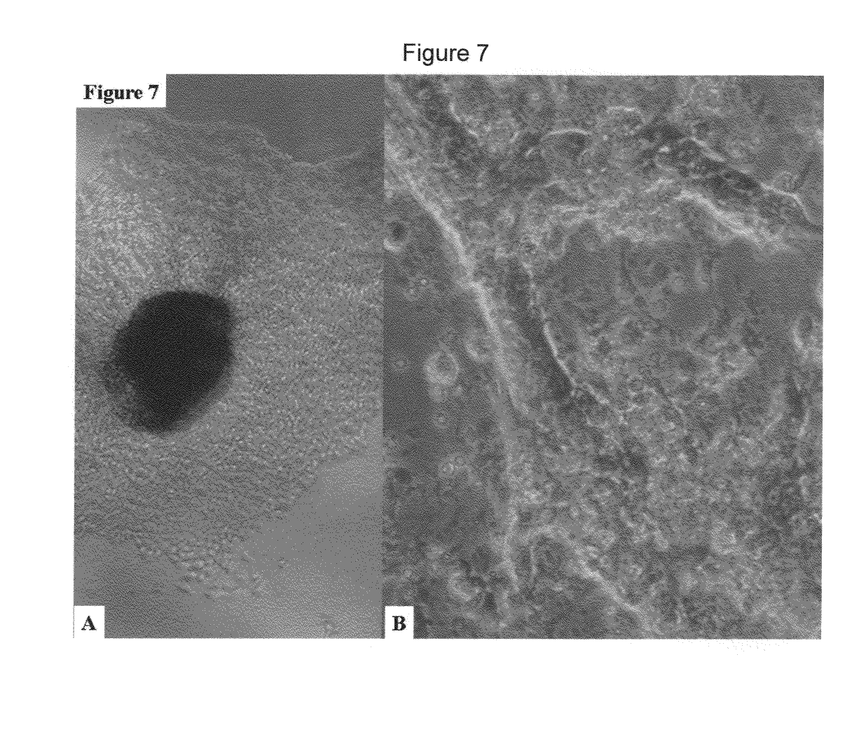

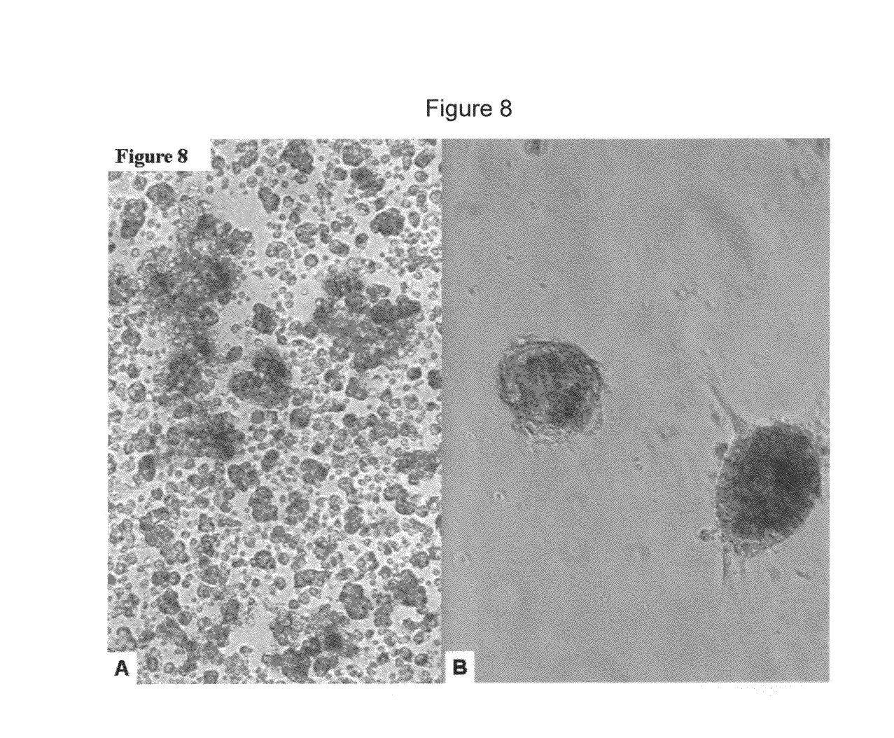

FIGS. 5A-C are graphical comparisons of CK19, albumin and AFP RNA levels normalized to that for the GAPDH housekeeping gene in hepatic progenitors cultured in HA hydrogel hydrogels, in hepatic stem cells cultured on plastic and in hepatoblasts freshly isolated from fetal liver cell suspensions. For each 30 ng of total RNA from freshly isolated hepatoblasts, there were high levels of AFP (130 strands), albumin (7000 strands), and relatively low levels of CK19 (1.2 strands). By contrast, the RNA isolated from hepatic stem cells showed no AFP at all, low levels of albumin (2.6 strands) and high levels of CK19 (100 strands). The hepatic progenitors seeded into the HA hydrogels showed low levels of CK19 (1.66 strands), low but detectable levels of AFP (0.33 strands), and levels of albumin (5.77 strands) that are higher than that in the hepatic stem cells, but dramatically lower than that observed in the freshly isolated hepatoblasts. Cyp3A4, a P450 cytochrome found in mature hepatocytes, could not be detected in either the hepatic stem cells or in the hepatic progenitors maintained in the HA hydrogels. Thus, the hepatic progenitors in the HA hydrogels are not stem cells, since they express AFP and ICAM-1, but the quantitative levels of their functions are closer to the stem cells than to the freshly isolated hepatoblasts. In fact, these cells are early stage hepatoblasts.

TABLE 3
Primer Sequences used in the RT-PCR Assays

| | | | | Tₘ Forward/ | Product |
|---|---|---|---|---|---|
| | Gene Bank | Forward Primer | Reverse Primer | Reverse | length |
| Gene | Acc. No. | (5′→3′) | (5′→3′) | Primer (° C.) | (bp) |
| ALB | NM_000477 | gtgggcagcaaatgttgtaa | tcatcgacttccagagctga | 59.59/59.66 | 188 |
| AFP* | NM_001134 | accatgaagtgggtggaatc | tggtagccaggtcagctaaa | 59.64/58.53 | 148 |
| CK19 | NM_002276 | ccgcgactacagccactact | gagcctgttccgtctcaaac | 60.47/59.85 | 152 |
| GAPD | NM_002046 | atgttcgtcatgggtgtgaa | gtcttctgggtggcagtgat | 59.81/60.12 | 173 |
| C3A4 | NM_017460 | gcctggtgctcctctatcta | ggctgttgaccatcataaaagc | 57.11/60.86 | 187 |

*The AFP primers are ones to detect uniquely hepatic-specific AFP, as reported in U.S. Patent Application No. 20030148329, the disclosure of which is incorporated herein in its entirety by reference.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

What is claimed:

1. A method of maintaining cells ex vivo under conditions that are 3-dimensional (3-D) and that are permissive for long-term maintenance, for expansion, and/or for differentiation comprising:
(a) providing cells; and
(b) culturing the cells in serum-free culture medium and on a complex of hyaluronans with or without other extracellular matrix components and with or without hormones or growth factors to maintain, propagate and/or differentiate a population of cells.

2. The method of claim 1 in which the cells are hepatic stem cells.

3. The method of claim 1 in which the cells are hepatoblasts.

4. The method of claim 1 in which the cells are committed progenitors.

5. The method of claim 1 in which the cells are mature cells.

6. The method of claim 1 in which the hyaluornans are complexed with other extracellular matrix components and/or hormones or growth factors.

7. The method of claim 6 in which the extracellular matrix components are one or more collagens (e.g. type III collagen), one or more basal adhesion molecules (e.g. laminin), one or more proteoglycans or their glycosaminoglycan chains (e.g. heparin proteoglycan) or a mixture thereof.

8. The method of claim 5 further comprising one or more hormones.

9. The method of claim 8 in which the hormones are insulin, transferrin/fe, tri-iodothyronine, T3, growth hormone, glucagon, or combinations thereof.

10. The method of claim 5 further comprising one or more growth factors.

11. The method of claim 10 in which the growth factors are epidermal growth factor (EGF), a fibroblast growth factor (FGF), an interleukin, a leukemia inhibitory factor (LIF), a transforming growth factor-β (TGF-β), or combinations thereof.

12. The method of claim 11 in which the interleukin is IL-6, IL-11, IL-13), or combinations thereof.

13. The method of claim 1 in which the hyaluronans are chemically cross-linked.

14. The method of claim 13 in which the hyaluronans are chemically cross-linked through aldehyde bridges.

15. The method of claim 13 in which the hyaluronans are chemically cross-linked through disulfide bridges.

16. The method of claim 15 in which the extracellular matrix comprising hyaluronans cross-linked through disulfide bridges, called Extracell-LGTM

17. The method of claim 1 in which the extracellular matrix further comprises one or more specific collagens, one or more specific isoforms of basal adhesion molecules, one or more species-specific or tissue-specific proteoglycans or their glycosaminoglycan chains, one or more hormones, and/or one or more growth factors, or mixtures thereof.

18. The method of claim 1 in which the cells are obtained from liver.

19. The method of claim 1 in which the cells are adult liver cells

20. The method of claim 18 in which the liver is fetal liver

21. The method of claim 18 in which the liver is neonatal liver

22. The method of claim 18 in which the liver is pediatric liver

23. The method of claim 18 in which the liver is adult liver

24. The method of claim 1 in which the serum free culture medium comprises insulin, transferrin, or both.

25. The method of claim 1 in which the serum free culture medium consists essentially of insulin, transferrin, lipids, calcium, zinc and selenium.

26. The method of claim 1 in which the serum free culture medium consists essentially of insulin, transferrin, lipids, calcium, zinc and selenium.

27. The method of claim 1 in which the serum free culture medium is further free of any growth factors or hormones other than insulin and transferrin.

28. A method of propagating cells ex vivo comprising:
(a) providing cells;
(b) culturing the cells in serum-free culture medium and on hyaluronans to enable long-term survival, expansion and/or differentiation of a population of cells.

29. The method of claim 28 in which the cells are stem cells.

30. The method of claim 28 in which the cells hepatoblasts.

31. The method of claim 28 in which the cells are committed progenitors.

32. The method of claim 28 in which the cells are mature hepatocytes or biliary cells.

33. The method of claim 28 in which the extracellular matrix further comprises one or more collagens, one or more basal adhesion molecules, one ore more proteoglycans or their glycosaminoglycan (GAG) chains, one or more hormones, one or more growth factors, or combination thereof.

34. The method of claim 33 in which the collagen is a type, I, III, IV or V collagen.

35. The method of claim 33 in which the basal adhesion molecule is an isoform of laminin or fibronectin or both.

36. The method of claim 33 in which the proteoglycans/GAG is a heparin, a heparin proteoglycans, chondroitin sulfate/chondroitin sulfate proteoglycans, dermatan sulfate/dermatan sulfate proteoglycans, heparan sulfate/heparan sulfate proteoglycans, or combinations thereof.

37. The method of claim 28 in which the hyaluronans are chemically cross-linked.

38. The method of claim 37 in which the hyaluronans are chemically cross-linked through aldehyde bridges.

39. The method of claim 37 in which the hyaluronans are chemically cross-linked through disulfide bridges.

40. A composition comprising a cell culture of cells, serum-free culture medium, and an extracellular matrix complex comprising hyaluronans.

41. The method of claim 40 in which the cells are stem cells.

42. The method of claim 40 in which the cells hepatoblasts.

43. The method of claim 40 in which the cells are committed progenitors.

44. The method of claim 40 in which the cells are mature hepatocytes or mature biliary epithelial cells

45. The method of claim 40 in which the extracellular matrix further comprises one or more collagens, one or more basal adhesion molecules, one or more proteoglycan(s) or its/their GAG chain, one or more hormones, one or more growth factors, or combination thereof.

46. The method of claim 45 in which the collagen is type III collagen.

47. The method of claim 45 in which the basal adhesion molecule is laminin.

48. The method of claim 45 in which the proteoglycans/GAG is a heparin or a heparin proteoglycan

49. The method of claim 40 in which the hyaluronans are chemically cross-linked.

50. The method of claim 49 in which the hyaluronans are chemically cross-linked through aldehyde bridges.

51. The method of claim 49 in which the hyaluronans are chemically cross-linked through disulfide bridges.

52. A container for propagation of hepatic progenitors comprising:
(a) a container, and
(b) an insoluble material comprising hyaluronans and at least one other extracellular matrix component selected from the group consisting of collagen, basal adhesion protein, proteoglycans or their glycosaminoglycan chains, hormone, and growth factor, wherein the insoluble material is present in suspension within the container or substantially coats at least one surface of the container.

53. The container of claim 52 in which the container is a tissue culture plate, a bioreactor, a lab cell or a lab chip.

54. The container of claim 52 in which the collagen is collagen type I, III, IV, V, VIII, XII, XIII, or combinations thereof.

55. The container of claim 52 in which the basal adhesion protein is an isoform of laminin or fibronectin.

56. The container of claim 52 in which the glycosaminoglycan is heparan sulfate, heparin, chondroitin sulfate, dermatan sulfate, or combinations thereof.

57. The container of claim 52 in which the glycosaminoglycan chains of a proteoglycan are heparan sulfate-PG, heparin-PG, chondroitin sulfate-PG, dermatan sulfate-PG, or combinations thereof.

58. The container of claim 52 in which the hormone is insulin, transferrin/fe, growth hormone, tri-iodothyronine, glucagon, or combinations thereof.

59. The container of claim 52 in which the growth factor is an isoform of epidermal growth factor (EGF), an isoform of fibroblast growth factor (FGF), an isoform of transforming growth factor-β (TGF-β), an isoform of hepatocyte growth factor (HGF), an isoform of leukemia inhibitory factor (LIF), interleukin 6 (IL6), interleukin 11 (IL11), interleukin 13 (IL13), oncostatin M, or combinations thereof.

60. The container of claim 58 in which the glucocorticoid is hydrocortisone.
